(19) United States
(12) Patent Application Publication
(10) Pub. No.: US 2015/0133945 A1
(43) Pub. Date: May 14, 2015

(54) HANDHELD TRACKING SYSTEM AND DEVICES FOR ALIGNING IMPLANT SYSTEMS DURING SURGERY

(71) Applicant: Stryker Global Technology Center, Gurgaon (IN)

(72) Inventors: Anantharaman Dushyant, Delhi (IN); Ketan Jajal, Vallabh Vidyanagar (IN); Sachin Singhal, Rewari (IN); Hitesh Ganjoo, Gurgaon (IN); Diksha Babhoota, Delhi (IN); Dinesh Sharma, Faridabad (IN); Vijay Chandra Bose, Chennai (IN); Adam Bastian, Chester, NY (US)

(21) Appl. No.: 14/397,908

(22) PCT Filed: May 1, 2013
(86) PCT No.: PCT/IB2013/053441

§ 371 Date: Oct. 30, 2014

(30) Foreign Application Priority Data
May 2, 2012 (IN) 1345/DEL/2012
Oct. 25, 2012 (IN) 3283/DEL/2012

(51) Int. Cl. A61B 19/00; A61B 17/16; A61F 2/46

(52) U.S. Cl. CPC A61B 19/5244; A61F 2/4657; A61B 17/1666; A61B 19/56; A61B 19/54; A61B 19/38; A61B 2019/5257; A61B 2019/5265; A61B 2017/00203
USPC 606/102

(57) ABSTRACT

The present invention discloses handheld tracking systems and devices comprising of at least one handheld tracking device for intra-operative aligning or positioning of surgical implant systems and instruments with reference to the anatomy of a patient. The handheld tracking system further comprises of at least one trackable element. The handheld device is mounted at the proposed implantation site using the holding means while trackable element(s) is/are mounted at predetermined location(s) such that data from said deployed trackable element(s) relating to the position of the patient and the surgical instruments are input continuously into the handheld devices. The handheld device then processes the data on the basis of pre-loaded preoperative scanned images in the processing means to monitor the accurate placement of said implant system onsite in sterile environment.

### FIELD OF INVENTION

The present invention relates to handheld tracking system and devices for aligning implant systems during surgery and the process of performing minimally invasive surgical tracking with the said handheld tracking devices.

This complete specification is cognate of provisional application numbers 1345/DEL/2012 dated 2 May 2012 and 3283/DEL/2012 dated 25 Oct. 2012 under Section 9(2) of The Patents Act 1970.

### BACKGROUND

Over time and through repeated use, bones and joints can become damaged or worn. The cartilage in the joint areas which provides a cushioning effect on the joints during physical activity can become worn out or damaged due to repetitive strain on bones and joints as in case of athletic activity or when subjected to traumatic events, or due to certain diseases such as osteoarthritis. This condition results in pain, stiffness, and decreased mobility for the patient.

Arthroplasty procedure is commonly used to replace a diseased joint with an artificial joint. During a typical arthroplasty procedure, an arthritic or otherwise dysfunctional joint is reshaped and/or realigned, and an implant system is accurately positioned into the damaged region. Arthroplasty procedures may take place on any of the joints in different regions of the body including but not limited to knee, hip, shoulder and elbow.

One of the key goals of arthroplasty is restoration of the patient anatomy, specifically aligning or the positioning of implant components with reference to the anatomy. This alignment of the implant with the anatomy is usually performed by using mechanical instruments and/or navigation systems. The disadvantages of using mechanical instruments lie in probability of the inaccurate alignment since these are based on average anatomical relationships and do not take into account any parameter of that particular patient or surgical setup. Also, these mechanical instruments may not be very intuitive and easy to use, requiring large surgical incision as well as continuous monitoring of instruments, while manually aligning the instruments with the key anatomical landmarks on the body. These anatomical landmarks could be unstable resulting in in-accurate judgment.

Further the existing navigation systems in the arthroplasty procedure are very complex and expensive. Using such systems typically requires an additional person to handle the navigation computer outside the sterile area in the operation theater. Besides, the existing navigation systems depend on direct line-of-sight between external cameras or infra-red sensors placed outside the sterile environment, and navigation instruments used inside the sterile zone in the surgery. This makes the surgical procedure very cumbersome and requires the surgeon to frequently move out of the surgery site so that the camera system present outside the sterile area can see the tracker system present inside the sterile area

Therefore, there is a need of handheld tracking system and devices for intra-operatively aligning implant systems on a patient which are simpler to use during surgical procedures yet highly accurate, and which provide greater intra operative flexibility to the surgeon such that it is completely controlled and used inside the sterile field.

Above disadvantages have been addressed in one such article titled “Improving acetabular cup orientation in the total hip arthroplasty by using smartphone technology” published in the journal of the arthroplasty discloses the use of smartphone for the acetabular placement. The advantages of the use of smartphone assisted surgery are many; for instance accurate digital inclination, less time consumption, no need of external references incisions, and minimum set up requirements. However this article does not discloses information related to accurate detection of anteversion orientation, which is the critical angle. Any inaccuracy in anteversion orientation significantly increases the chances of post-surgery complications such as repeated dislocation.

### OBJECT OF THE INVENTION

It is an object of the present invention to provide handheld tracking systems and devices for intra-operatively aligning implant systems and/or surgical instrument with reference to the anatomy of the patient during surgery.

It is another object of the present invention to provide simple, easy to set up handheld tracking systems and devices for accurate positioning and/or sizing of implant system with reference to the anatomy of the patient during surgery and without being dependent on the line-of-sight connectivity outside the sterile area.

It is yet another object of the invention to provide active handheld device capable of indicating the position and/or size information through audio and/or visual means during surgical procedures.

It is yet another object of the invention to provide handheld tracking system comprising of at least one handheld device having sensors capable of detecting and displaying runtime implant system information within sterile environment.

It is yet another object of the invention to provide handheld device which can be mounted on multiple types of instruments during surgery as well as anchored on to the patient.

It is yet another object of the invention to provide handheld device with the capability of reading and processing pre-operative image based patient data as well as intra-operative images from implant systems, instruments and/or patient position.

It is yet another object of the invention to provide handheld device capable of communicating wirelessly using data communication link or protocol without any external device or reference.

It is yet another object of the invention to provide a method of handheld surgical tracking using the systems and devices of the present invention.

It is yet another object of the invention to provide cost effective and less cumbersome tracking system which can easily be used by the surgeon during various surgical procedures.

### SUMMARY OF THE INVENTION

The present invention relates to handheld tracking systems and devices comprising of at least one handheld tracking device for intra-operative aligning or positioning of surgical implant systems and instruments with reference to the anatomy of a patient. The present invention also provides process of minimally invasive surgical tracking within the sterile environment using the systems and devices of the present invention

Accordingly, the instant invention provides handheld tracking system for intra-operative alignment of surgical instruments and/or implant system. The handheld tracking system comprises of at least one handheld device which has means for input of data, processing means, storage means and means for output, of processed data, rechargeable source of power. The handheld tracking system further comprises of at least one trackable element, and at least one holding means, which is used for securing the handheld device in sterile environment.

The handheld device loaded with pre-operative scan data and other patient specific data, is mounted at the proposed implantation site with the help of holding means while trackable element(s) are mounted at predetermined location(s). The data from the deployed trackable element(s) relating to the position of the patient and the surgical instruments are input continuously to the handheld device through the means for input, and further continuously processed on the basis of the pre-loaded preoperative scanned images in the processing means, to provide continuous processed data to monitor the accurate placement of the implant system onsite in sterile environment.

In another aspect of invention, present invention also discloses the method of using handheld tracking system. The method may comprise the step of uploading patient specific preoperative scans and patient specific data in the handheld device. Additionally, method may include the uploading position data of the trackable element positioned on the head of femur to said handheld device to prevent any length discrepancy in leg length of the patient on completion of surgery. The method further may comprise the step of mounting of handheld device on to the patient anatomy/bone and securing the same with the holding means. The method may include positioning of at least one trackable element on to the surgical instrument, aligning the patient with preoperative scan stored on to the handheld device to determine the desired position of the implant system and then dynamical tracking the surgical instrument in relation to the identified reference position to ascertain the correct course of surgery and the subsequent implantation of the implant system

### BRIEF DESCRIPTION OF THE DRAWINGS

FIG. 1 shows the schematic illustration of handheld tracking system in which features of the present invention may be implemented.

FIG. 2 shows the front view of handheld device depicting different features of the handheld device.

FIG. 3 shows the front view of handheld device, particularly displaying the information pertaining to relative measurements.

FIG. 4 shows the rear view of the handheld device with its features.

FIG. 5 shows the mounting arrangement of handheld device on to a patient.

FIG. 6 shows the securing mechanism/secured fixing of handheld device in device holder, depicting features of the device holder.

FIG. 7 shows the implementation/arrangement of various features of handheld tracking system on to a patient.

FIG. 8 shows the implementation of handheld tracking system and devices, more particularly the relative arrangement of handheld device 1 and 2 on to a patient.

FIG. 9 depicts anatomically matched block for fixing the initial position of the surgical instrument/implant in the handheld device.

FIG. 10 shows the illustration of performing reaming step to a desired depth in an arthroplasty surgery

FIG. 11 shows the illustration of implant system alignment to obtain a desired leg length in an arthroplasty surgery

FIG. 12 shows the process flow diagram illustrating the use of the handheld tracking system during surgery.

### DETAILED DESCRIPTION OF THE INVENTION WITH REFERENCE TO THE DRAWINGS AND ILLUSTRATED WITH EXAMPLES

The present invention obviates the aforesaid problems in the prior art and provides handheld tracking systems and devices for aligning implant system during surgery. The said handheld tracking system comprises of handheld device for intra-operatively aligning implant system and/or surgical instrument with reference to anatomy of the patient during arthroplasty. The system of the present invention is used to determine accurate positioning and/or size of implant system with reference to anatomy of the patient during surgery. As used herein the terms “arthroplasty or arthroplasty procedures” refers to an operative procedure of orthopedic surgery, in which an arthritic or otherwise dysfunctional joint is reshaped and/or realigned and optionally an implant system is positioned into the damaged region. Arthroplasty procedure may take place on any of the joints in different regions of the body including but not limited to knee, hip, ankle, shoulder, and elbow. The term “holding means” refers to means for securing handheld device to the anatomy of the patient or to the surgical instrument in sterile environment.

In case of arthroplasty procedure accurate positioning of implant is very critical. Any incorrect positioning of implant may lead to severe post-operative discomfort such as dislocation of joint, poor loading condition, thereby resulting in the failure of the implant system and requiring further expensive and extremely skilled correction/revision surgery by expert surgeons. The present invention discloses a system comprising of at least one handheld device. The handheld device is capable of being connected to an anatomical part of body of a patient by holding means as well as it can be attached to an instrument to be used by the surgeon during the arthroplasty procedure wherein at least one of the handheld devices is capable of indicating the position and/or size information through audio and/or visual means.

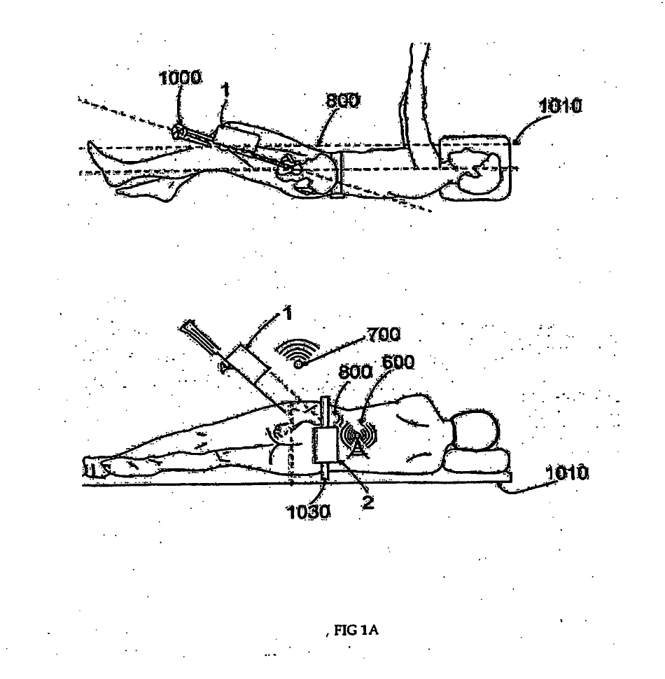

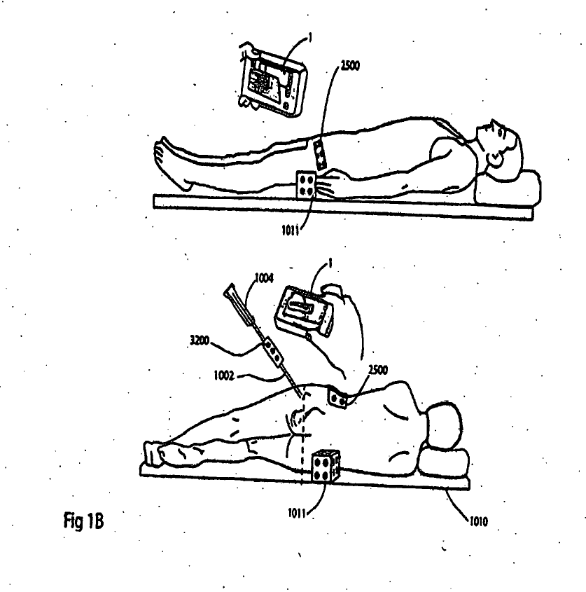

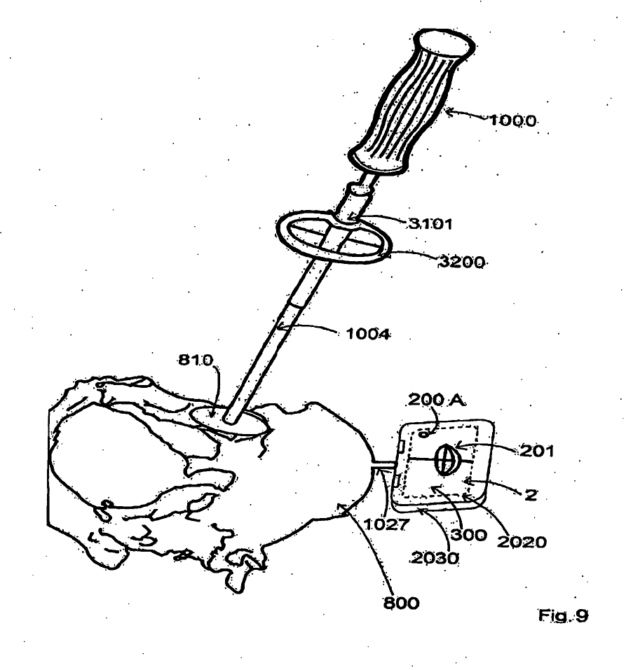

FIG. 9 shows the handheld tracking system comprising of one handheld device while FIGS. 1, 7, 8 illustrates the handheld tracking systems having two handheld devices (1, 2).

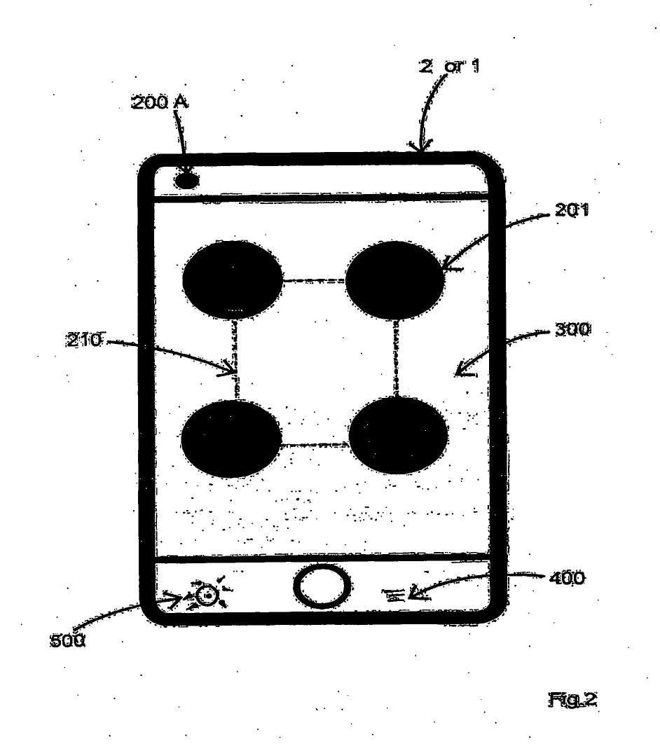

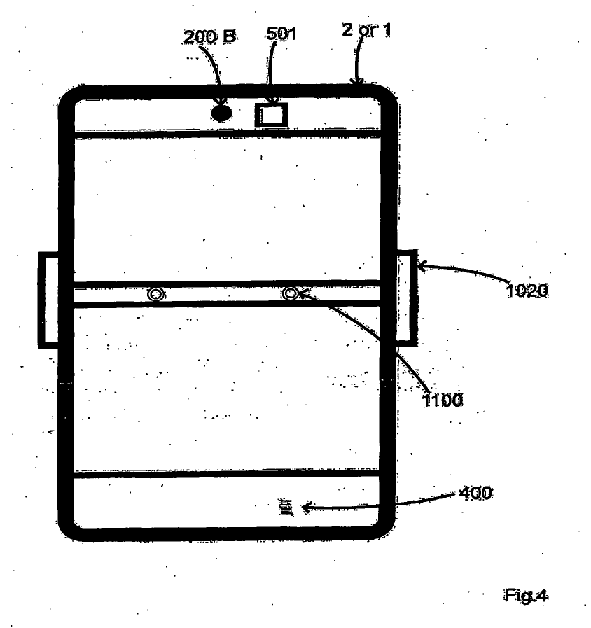

FIG. 2, 3 and FIG. 4 depict detailed illustration of handheld tracking device. FIG. 9 and FIG. 10 and FIG. 11 depict the method of use of handheld tracking system in arthroplasty surgery.

The handheld tracking system as shown in FIG. 9 comprising of one handheld device (2) and trackable element (3200). The handheld device is mounted on the anatomy of patient body (800) by holding means (1027) while trackable element (3200) which is readable by said handheld device is mounted on the surgical instrument (1004) through slot (3101). The said trackable element (3200) may be either made of reflected material or have unique colors. The trackable element (3200) is secured on to the surgical instrument (1004) through means (3101) in such a manner that device (2) can easily detect the trackable element (3201).

The handheld device may comprise of processors, rechargeable or single use battery, speakers, touch screen displays, camera sensors, accelerometer sensors, gyro sensors, flashlights, etc. Said sensors present in the handheld device are capable of communicating with each other or with the surgical team without any external device or reference and are capable of detecting and displaying runtime implant system information with-in the sterile environment.

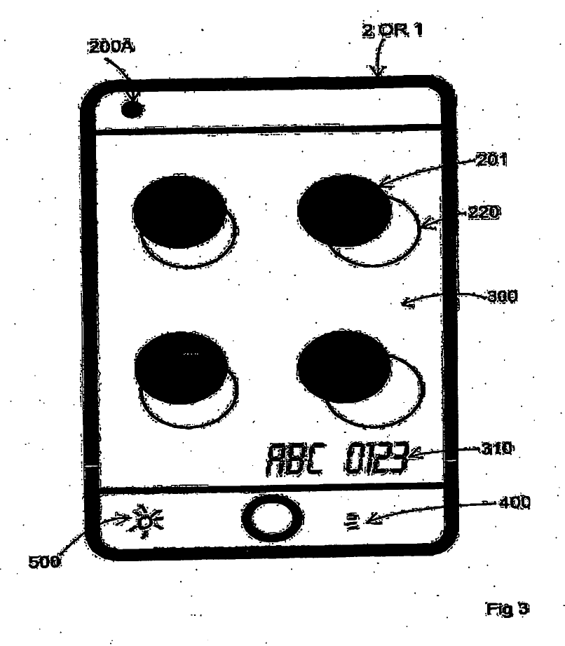

FIGS. 2, 3 and 4 are directed towards the detailed illustrations of exemplary handheld tracking device. The said handheld device comprises of display screen (300), digital cameras (200A, 200B) on the front and rear side of handheld device respectively, built in speakers (400), built in flashlights (500, 501) on the front and rear side of the handheld device respectively. The handheld device also comprises of digital memory in order to store the preoperative as well as intra-operative data that are useful in the various stages of surgeries.

The digital cameras (200A, 200B) have sensor(s) for capturing image, processor(s) to process the captured image and results are displayed on the said display screen (300). The display screen can also be a touch screen which can be used as input to record the initial desired position of the trackable element (201) by the surgeon. The display screen displays initial (201) as well as current dynamic position (220) of trackable element. The handheld device recognizes the tracker shape and using image processing displays the boundary of the tracker system (210) which may be circle(s), square(s) or ellipse(s). The display screen may also provide the information indicating the relative difference (310) between said initial and current position of trackable element in reference to a specific surgical step in surgery. The said information is quite useful in surgical procedure, for example in total hip arthroplasty, as it indicates the position of acetabular cup, anteversion, inclination, depth of reaming etc.

The built-in speaker (400) of the said handheld device (1, 2) can provide specific feedback during surgery such as confirmation sound or out of limit “warning beep”. The built-in flash lights (500, 501) with the built-in cameras (200A, 200B) can accurately capture trackable element (2500, 3200, 3300, 3500) positions even with a complex background.

In the arthroplasty surgery the systems and the devices of the present invention can be easily placed in a sterile zone. FIG. 4 describes the device holder to hold the handheld device (1, 2), inside the sterile environment. The said device holder (1020) may include reusable or disposable transparent sterile bag.

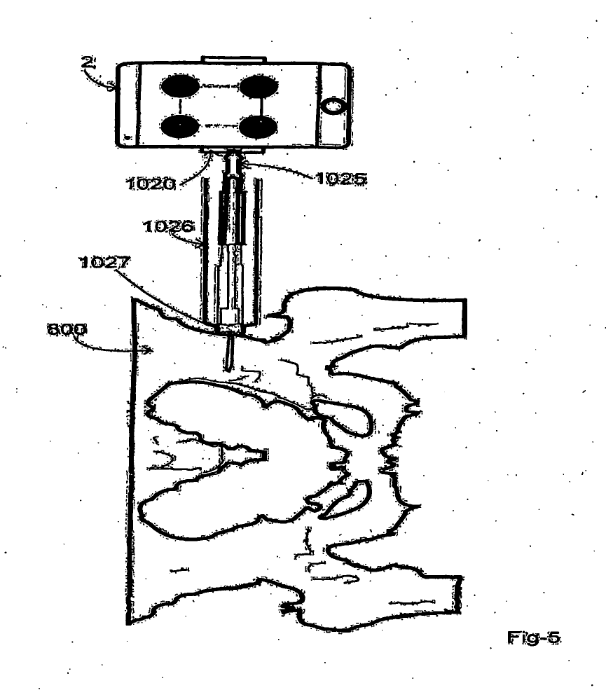

FIG. 5 depicts the mounting arrangement (1025, 1026, 1027) of handheld device (2) on to the anatomy or bone of the patient (800). It comprises of assembly of instrument (1025), sterile sleeve (1026) and sterile pin (1027).

FIGS. 5 describes holding means of handheld device (2) on to the patient's anatomy. The one side of said sterile pin or screw (1027) is connected to the patient's anatomy or bone (800) while the other side is connected to device holder (1020). The sterile pin or screw (1027) is kept inside the sterile sleeve (1026) such that the sterile pin can be inserted into the patient bone (800) even in the non-sterile environment, thereby allowing the surgeon to position the device at any desired location prior and/or during the surgery.

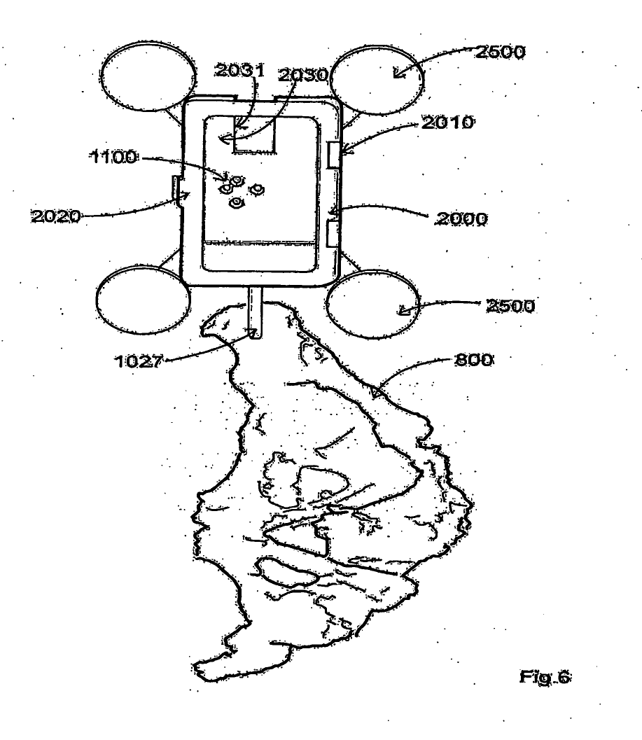

FIG. 6 also describes another such holding mechanism of handheld tracking device (2) on to the patient's anatomy. The said device holder (2000) consists of a base (2010) which is shaped to match the handheld device (2) for a secured fit (2030). The said device holder (2000) also has a door like mechanism (2020) such that it can hinge with the said base of device holder (2010) to form a close and secure enclosure thereby protecting the handheld device (2). The base of the device holder (2010) may have an opening (2031) to ensure the proper functioning of the camera/sensors of the hand held device (2). The handheld (2) device could be mounted onto the bone via assembly of device holder (2000) and the pin (1027) secured through (1100) screw mechanism.

FIGS. 1A, 1B, 7 and 8 depicts the handheld tracking system and devices in sterile environment in which two hand held devices (1, 2) are illustrated. The handheld device (1) is secured on the surgical instrument (1000, 1003) while other handheld device (2) is mounted on the patient's anatomy (800). The handheld device (2) is secured to the anatomy or bone of the patient via holding device (1030) or assembly of (1020-1025-1026-1027 or 1027-2000). The patient is positioned on the flat surface (1010) of the surgical table which may have grids and/or marks to facilitate patient positioning. The bidirectional data communication happens between device 1 channel (600) and device 2 channel (700)

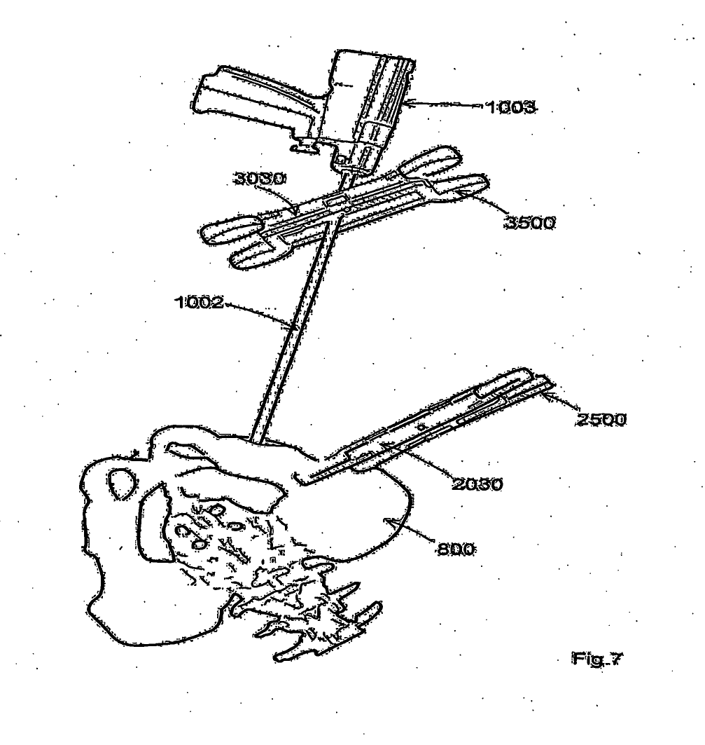

FIG. 7 illustrates the arrangement of at least one of the trackable elements (3500, 2500) in same or different planes on their respective handheld devices (1, 2). The trackable elements are made of either reflective material or have unique color enabling camera sensor to easily identify the elements. FIG. 7 also shows the axis (1002) of the surgical instrument (1003) whose position is to be tracked during the surgery relative to patient anatomy (800) for accurate surgical outcome.

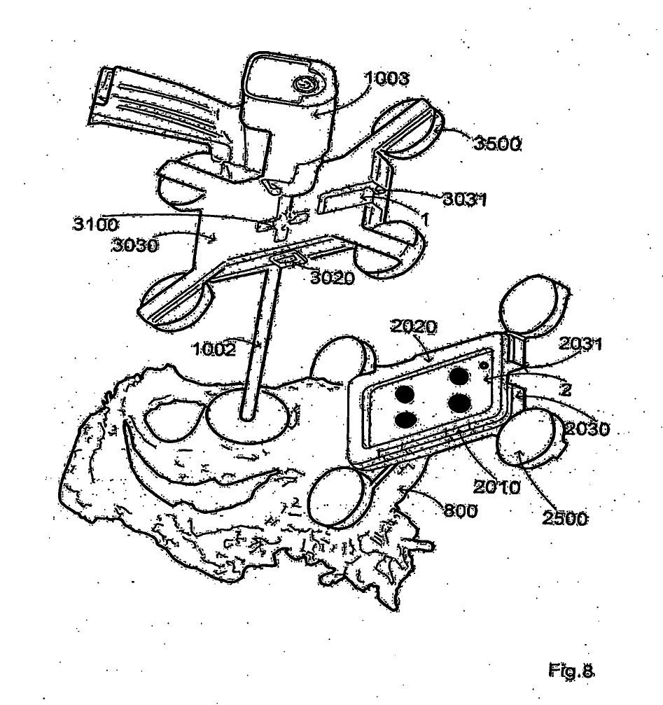

FIG. 8 shows another detailed view of the arrangement described in FIG. 7. It shows device holder (3000) similar in construction to device holder (2000 and/or 1020). The said device holder (3000) of the handheld device (1) has a door like mechanism (3020) such that it can hinge with the said base of device holder (3030) to form a close and secure enclosure thereby protecting the handheld device (1). The base of the device holder (3030) may have an opening (3031) to ensure the proper functioning of the camera/sensors of the handheld device (1).

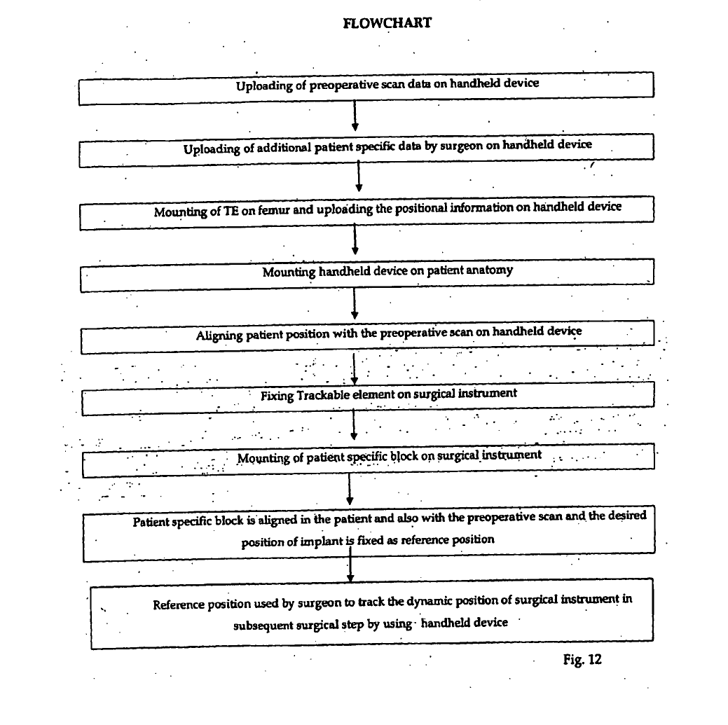

FIG. 12 shows in detail preferred method of using handheld tracking system which includes the steps of uploading of patient specific preoperative scans in the handheld device (1,2), uploading of additional patient-specific data by the surgeon in said handheld device, uploading position data of the trackable element (2700) positioned on the head of femur to said handheld device to prevent any length discrepancy in leg length of the patient on completion of surgery, mounting of handheld device on to the patient anatomy/bone and securing the same with said holding means, positioning of at least one trackable element (2500, 3200, 3300,3500) on to the surgical instrument, aligning the patient with preoperative scan stored on to said handheld device to determine the desired position of the implant system mounting of anatomically matched patient specific block (810) on to the surgical instrument, aligning it to the preoperative scan and fixing the desired position of implant as reference position on said display means of said handheld device, dynamically tracking the surgical instruments in relation to said identified reference position to ascertain the correct course of surgery and the subsequent implantation of the implant system

### EXAMPLES

In an exemplary embodiment, the instant invention provides the use of handheld tracking system and devices for hip arthroplasty. Hip arthroplasty is a surgical procedure in which the hip joint is replaced by a prosthetic implant system, which includes a cup placement on the acetabulum (socket) side and step placement on the femur (ball) side. Hip arthroplasty can be performed as a total replacement or a hemi (half) replacement. Such arthroplasty procedure is generally conducted to relieve arthritic pain or to fix severe physical joint damage as part of hip fracture treatment.

Generally the cup placement procedure is divided into two or three stages depending upon the type of implant used. In case of cemented cup implant, the acetabulum is reamed using a reamer as a first step and then a cup is placed along with cement. While in un-cemented press-fit implant, the acetabulum is reamed and then the cup is press-fitted onto the bone using an impaction tool.

The hip arthroplasty surgery usually starts with preoperative scan, which may be X-Ray, CT scan or MRI scans in order to assess the acetabular depth, size and anatomical landmarks. The preoperative scan also aids in the selection of the most favorable implant size suitable for the patient anatomy. After this step relevant patient data is transferred and/or stored into the handheld device.

During surgery, the handheld device (2) is mounted rigidly on to the pelvis bone (800) while trackable element (3200) is mounted on to the surgical instrument such as reamer as shown in FIG. 9. The camera sensor (200A) of the handheld device (2) captures the desired position of the trackable element (3200) and/or instrument axis (1002) of instrument (1003, 1004). This captured position could be optimized using either pre-op data, anatomically matched block (810) or surgeon's intra-operative clinical judgment. This captured information is processed and displayed (201) onto the screen (300) of the handheld device (2).

Once this desired position is approved by the surgeon, the handheld device guides the surgeon in the subsequent surgical steps to reproduce the planned/desired/approved instrument axis. For example, during the reaming procedure multiple sizes of reamers are used to prepare the acetabulum. The handheld device (2) displays the desired position as well as the current dynamic position of the trackable element (3200) and/or instrument (1004) as shown in FIG. 9. This allows the surgeon to correct the position of the surgical instrument and to go back to the pre-identified desired position repeatedly during the entire procedure of reaming and impaction, resulting in accurate acetabular implant component placement.

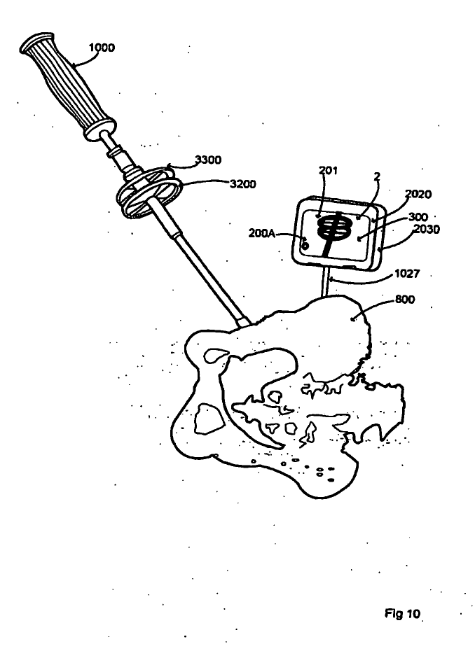

In another embodiment, the trackable element mounted on instrument (1004) has defined dimensions. The handheld device (2) is capable of processing the trackable element image (201) and recognizing it as object(s) with specific dimensions which may be in the form of circle(s), ellipse(s) or square(s) etc. This allows the handheld device (2) to indicate critical information such as angles, distances, diameters etc. For example, FIG. 10 shows two circular trackable elements (3200, 3300) of defined dimension that indicates depth of reaming.

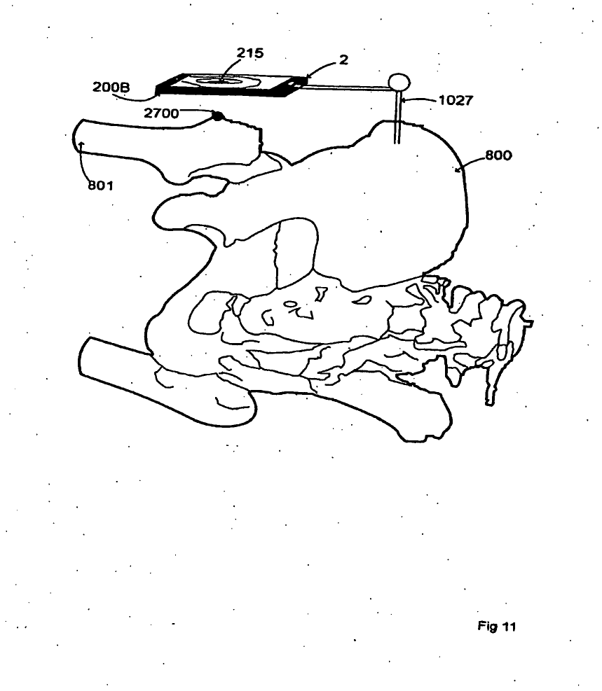

In yet another embodiment, the trackable element (2700) can be mounted onto one bone and the handheld device can be mounted onto another bone. The handheld tracking system in this situation can help accurately position the implant system and thereby also accurately align the relative position of the bones in a joint. For example, in hip arthroplasty procedure, FIG. 11 shows a simple trackable element (2700) which is affixed onto the femur bone (801) using a small button type tracker or simply by using methylene blue pen to mark a specific area during surgery. This trackable element/mark can be detected by the camera (200B) mounted on pelvis bone (800) and the processed image of this trackable element (215) is displayed onto handheld device (2). This trackable element position can be initially recorded by the surgeon before opening the joint. This position can be used at a later stage during stem and cup placement to accurately incorporate the leg length required for that patient.

Although the process described above is used for hip arthroplasty, the process of the present invention can be modified and adapted for other surgical procedures where recreation of patient anatomy is critical. Such surgical procedures may include, but are not limited to knee arthroplasty, hip arthroplasty, shoulder arthroplasty, wrist arthroplasty, ankle arthroplasty, spinal surgeries, and osteotomies.

The handheld tracking system of the present invention is able to accurately detect position of implant system and/or surgical instrument with respect to the native anatomy of the patient body without using any external reference from outside the sterile area. The handheld tracking system of the present invention does not require to be in the line-of-sight of any reference outside the sterile area.

Thus, the present invention provides simple, easy to set up, cost effective and less cumbersome surgical navigation systems and devices which can be easily used by a surgeon during various surgical procedures.

1. Handheld tracking system for intraoperative alignment of surgical instruments and/or implant system, said system comprising of;
at least one handheld device (1, 2), having means for input of data (200A, 200B, 300), processing means, storage means and means for output (300, 400) of processed data, rechargeable source of power, said handheld device being loaded with patient specific preoperative scanned images as well as other patient specific data,
at least one trackable element (1011, 2500, 3200, 3300,3500), mounted on predetermined locations
said handheld device capable of being placed at the proposed implantation site in a manner that all said trackable elements are capable of being monitored on said handheld device
such that data from said deployed trackable element(s) relating to the position of the patient and the surgical instruments is input continuously through the means for input, said data being processed on the basis of said pre-loaded preoperative scanned images and said patient specific information to provide continuous processed data to monitor the accurate placement of said implant system onsite in sterile environment.

2. Handheld tracking system as claimed in claim 1 wherein said means for input is a combination of devices selected from at least one digital camera (200A, 200B), multiple sensors, touch screen, touchpad, keyboard, voice input system.

3. Handheld tracking system as claimed in claim 1 wherein said means for output is a combination selected from display means, audio means, audiovisual means, touch screen which is capable of providing runtime information including initial input by surgeon, recording specific position and measure specific angle of said surgical instrument during various surgical steps.

4. Handheld tracking system as claimed in claim 2 wherein said multiple sensors includes inbuilt digital camera sensor, gyro meter sensor, accelerometer sensors.

5. Handheld tracking system as claimed in claim 1 wherein predetermined locations of the trackable elements are selected from the bone such as femur bone head and/or surgical instruments deployed in the surgery.

6. Handheld tracking system as claimed in claim 1 wherein said trackable element is selected from reflective material, material of unique color, methyl bluemarker, button and such other elements that allows said sensor to detect the position of said trackable element accurately.

7. Handheld tracking system as claimed in claim 1 wherein said handheld device is placed by itself at positions, or secured with holding means selected from holding device (1030), holding assembly (1025-1027-1030) with device holder (1020) having slots (1100), and sterile pin (1027) for fixing device holder (1020) having slots (1100).

8. Handheld tracking system as claimed in claim 7 wherein said holding assembly comprises of holding instrument (1025) that is connected to said device holder (1020), removable sterile sleeve (1026) and sterile pin (1027), such that said holding instrument (1025) is placed within said removable sterile sleeve (1026) and fixed on location using sterile pin (1027).

9. Handheld tracking system as claimed in claim 7 wherein said sterile pin (1027) directly connects said device holder (1020) of the handheld device to the bone of the patient.

10. Handheld tracking system as claimed in claim 7, wherein holding device (1030) rigidly connects the handheld device to the anatomy of the patient.

11. Handheld tracking system as claimed in claim 7, wherein device holder is sterile bag having slots.

12. Handheld tracking system as claimed in claim 1 wherein said handheld device has rechargeable battery.

13. Handheld tracking system for intraoperative alignment of surgical instruments and/or implant system as claimed in claim 1 wherein said system comprises of more than one handheld device (1, 2) each being capable of communicating wirelessly to the other using data communication link or protocol without any external device or reference.

14. The method of using handheld tracking system for aligning surgical instruments/implant system, said method comprising the steps of:
uploading patient specific preoperative scans in the handheld device (1,2),
uploading of additional patient-specific data by the Surgeon in said handheld device (1,2),
uploading position data of the trackable element (2700) positioned on the head of femur to said handheld device to prevent any length discrepancy in leg length of the patient on completion of surgery,
placing said handheld device near the patient or mounting said handheld device on to the patient anatomy/bone and securing the same with said holding means (1020, 1030, 1025, 1026, 1027),
positioning of at least one trackable element (200) on to the surgical instrument (1004),
aligning the patient on flat surface of trackable surgical table (1011) with preoperative scan stored on to said handheld device to determine the desired position of the implant system,
mounting of anatomically matched patient specific block (810) on to the surgical instrument, aligning it to the preoperative scan and fixing the desired position of implant as reference position (201) on said display means (300) of said handheld device,
dynamically tracking the surgical instruments in relation to said identified reference position to ascertain the correct course of surgery and the subsequent implantation of the implant system.
